(19) United States
(12) Patent Application Publication
(10) Pub. No.: US 2007/0016076 A1
(43) Pub. Date: Jan. 18, 2007

(54) Dermatone skin analyzer

(76) Inventor: Kambiz Youabian, Los Angeles, CA (US)

Correspondence Address: Youabian, Inc.;#400, 11022 Santa Monica Blvd., Los Angeles, CA, 90025, US

(21) Appl. No.: 11/183,572

(22) Filed: Jul. 18, 2005

(51) Int. Cl. A61B 6/00

(52) U.S. Cl. 600/476

(57) ABSTRACT

The Dermatone Skin Analyzer measures human skin tone darkness which is dependent on skin reflection properties and the concentrations of melanin, hemoglobin, and other pigments within the skin. The skin tone readings are used to assist in laser and light based treatments of the skin.

### BACKGROUND OF THE INVENTION

Laser and light based treatments of the skin for various dermatological and cosmetic conditions are influenced by the skin tone darkness. The settings that are used on the laser and light based treatments must be adjusted according to the skin tone darkness. Physicians, nurses, and technicians who operate such lasers and light based treatments have a difficult time in accurately assessing the skin tone darkness by simply looking at the skin. The Dermatone Skin Analyzer was invented to assist in such situations.

Skin tone is mainly influenced by the concentrations of melanin, hemoglobin, and various other skin pigments. Melanin, hemoglobin, and other skin pigments tend to absorb light energy at different wavelengths of light. The higher the concentrations of these structures within the skin, the more energy is absorbed by the skin. Laser and light based treatments are also influenced by the skin reflection properties. As the skin becomes more reflective, it absorbs less light and thus reflects more light. All of these factors combined will affect how much energy is absorbed by the skin during a laser or light based treatment.

### SUMMARY OF THE INVENTION

The Dermatone Skin Analyzer measures human skin tone darkness which is dependent on skin reflection properties and the concentrations of melanin, hemoglobin, and other pigments within the skin. The Dermatone Skin Analyzer uses a light emitting source to generate wavelengths of light within the spectrum of light visible to the human eye. The device is placed over the skin to be analyzed and the generated light is directed towards the skin through a hole at the tip of the device. A light sensor which is sensitive to the light within the emitted spectrum is used to measure the amount of light which is reflected from the skin. Based on the various wavelengths and intensities of light absorbed by the sensor, the device uses a digital circuit to analyze and convert the data into a number which correlates to the skin tone darkness. The digital circuit is comprised of electrical components and microchips which are programmed to perform the calculations. The device includes an LCD (liquid crystal display), which displays the readings and messages sent by the digital circuit.

The skin tone darkness will depend on various factors such as the concentrations of melanin and hemoglobin in the skin, as well as the skin's reflection properties. As the skin's concentrations of melanin and hemoglobin increase, the skin will absorb more light and thus reflect less light. The device analyzes and correlates the light measured by the sensor to display a number which correlates to the skin tone darkness. The readings are also influenced by the skin's reflection properties. As the skin becomes more reflective, it absorbs less light and thus reflects more light. The light sensor will detect the amount of light reflected by the skin and will calculate the skin tone darkness based on its programmed parameters. The calculated skin tone is then displayed to the user on the LCD display.

The device also utilizes a calibration function programmed into the digital circuitry. The calibration function allows the user of the device to calibrate the device on a regular basis to ensure accurate readings by the device. The calibration involves performing 5 consecutive measurements on a series of 5 images of different tones of darkness. The images are a standard set of images used for calibrating all manufactured devices. The digital circuitry of the device is preprogrammed with the tones of the calibration images and it calibrates its calculations based on the calibration results. The digital circuitry has an EPROM memory which remembers the results of the calibration even if the device is turned OFF.

The device is portable and is powered by a standard 9-volt battery which can be easily replaced. When the battery level is low and needs to be replaced, the device's digital circuitry will inform the user by displaying a message on the LCD display.

### DRAWINGS DESCRIPTIONS

FIG. 1: Schematics of Electronics Components

FIG. 2: Assembly of Electronics Components

FIG. 3: Layout of Digital Circuitry

FIGS. 4-10: Plastic Cover Design & Dimensions

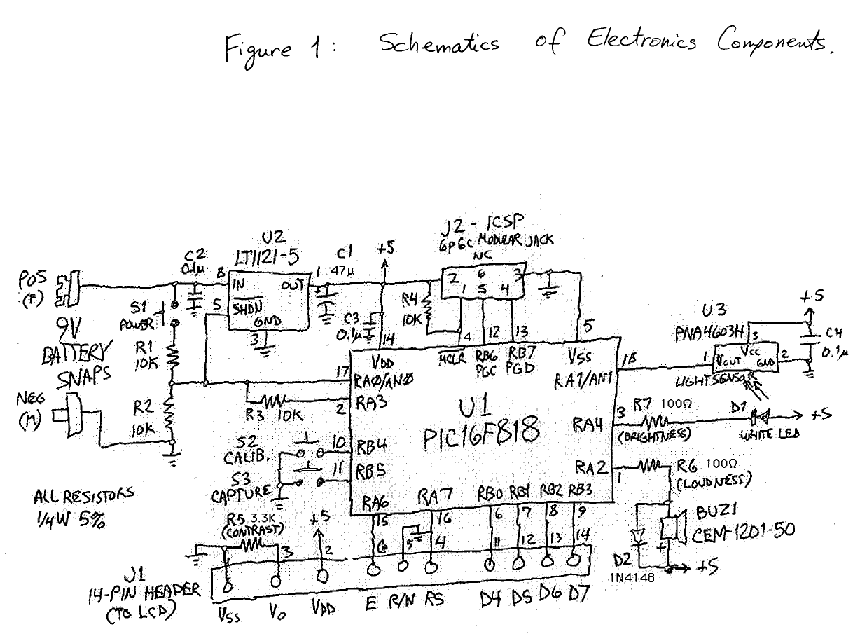

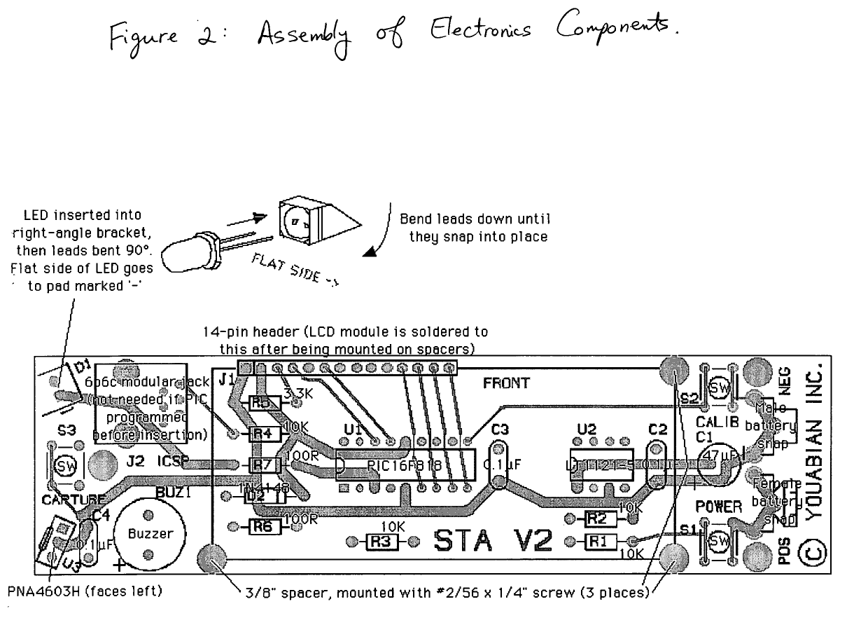

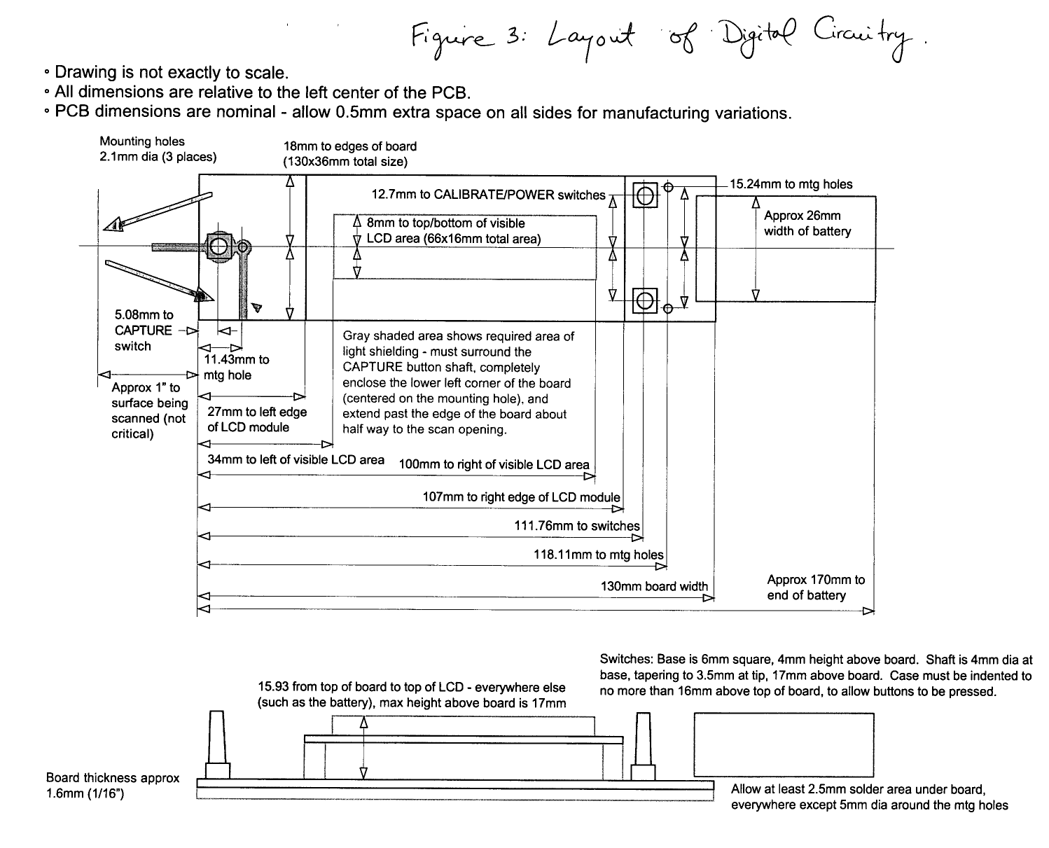

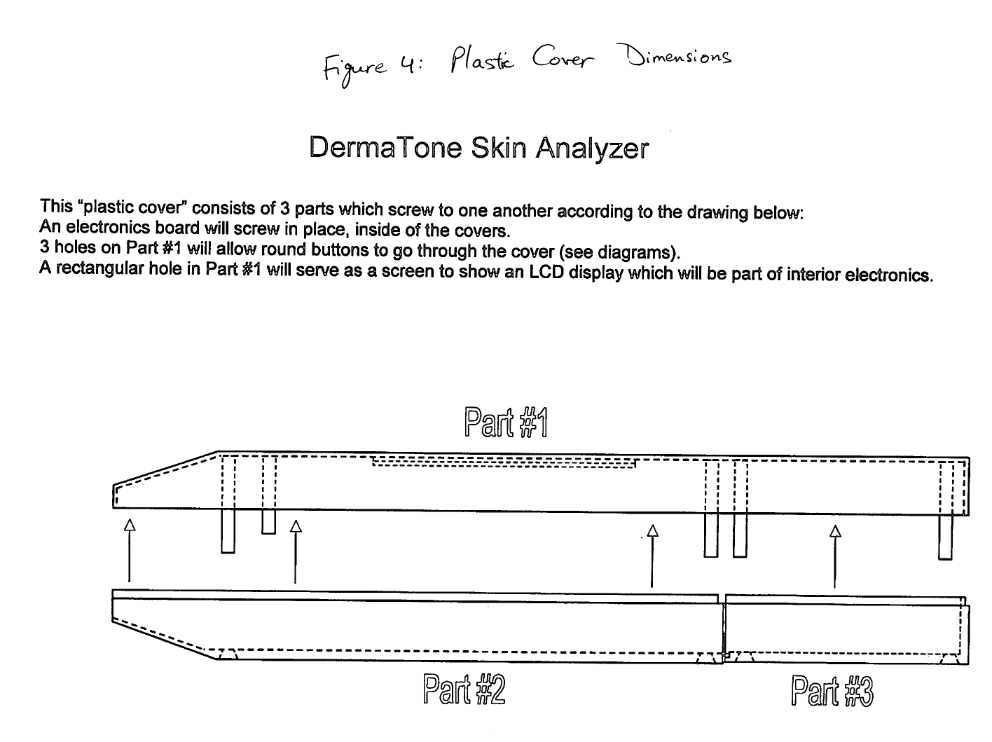

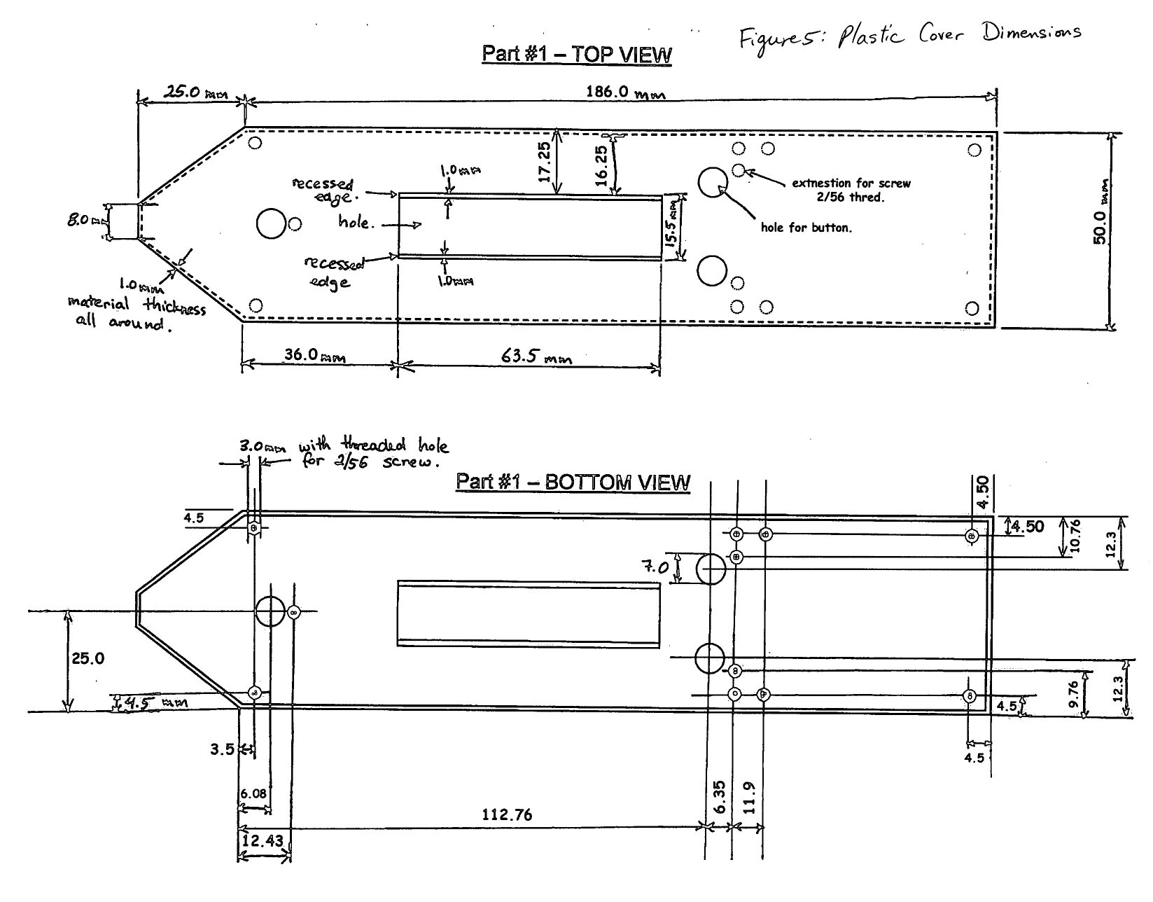

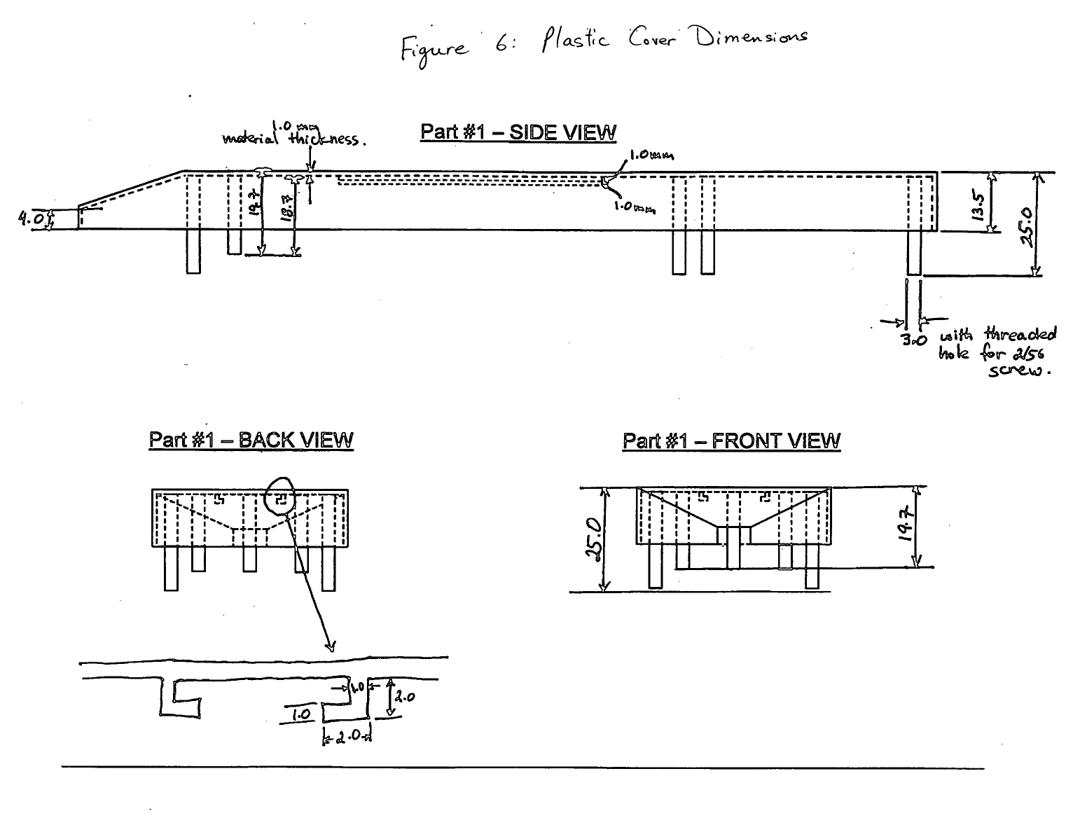

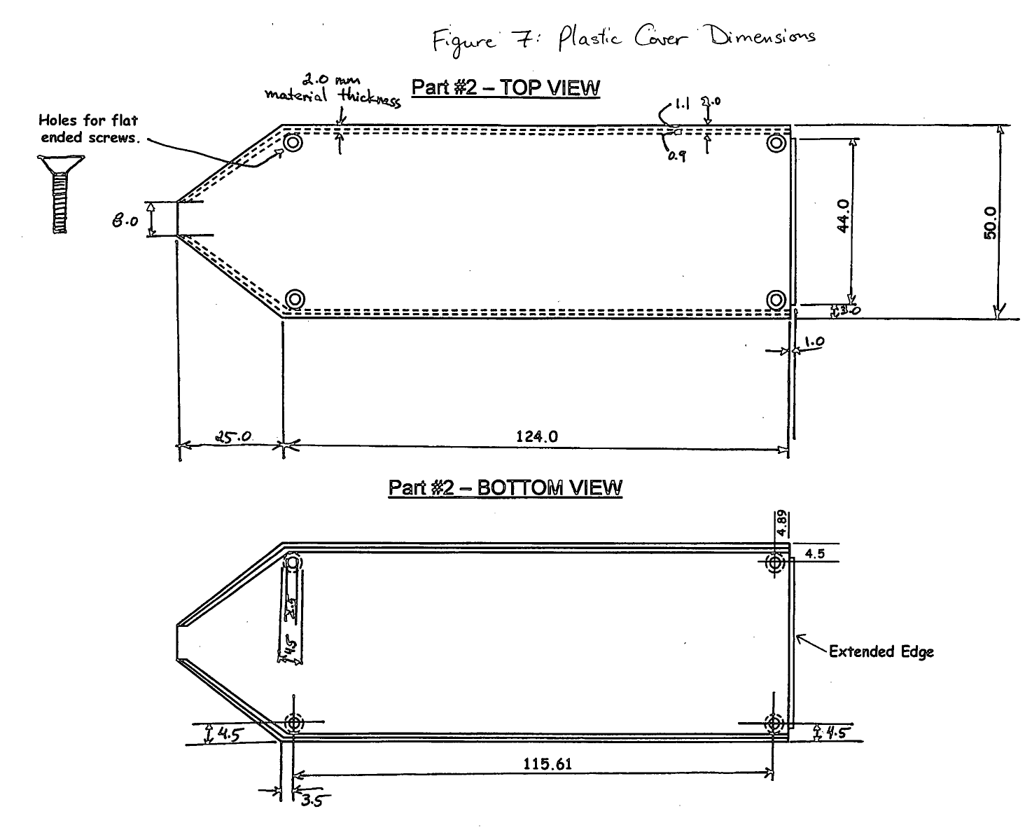

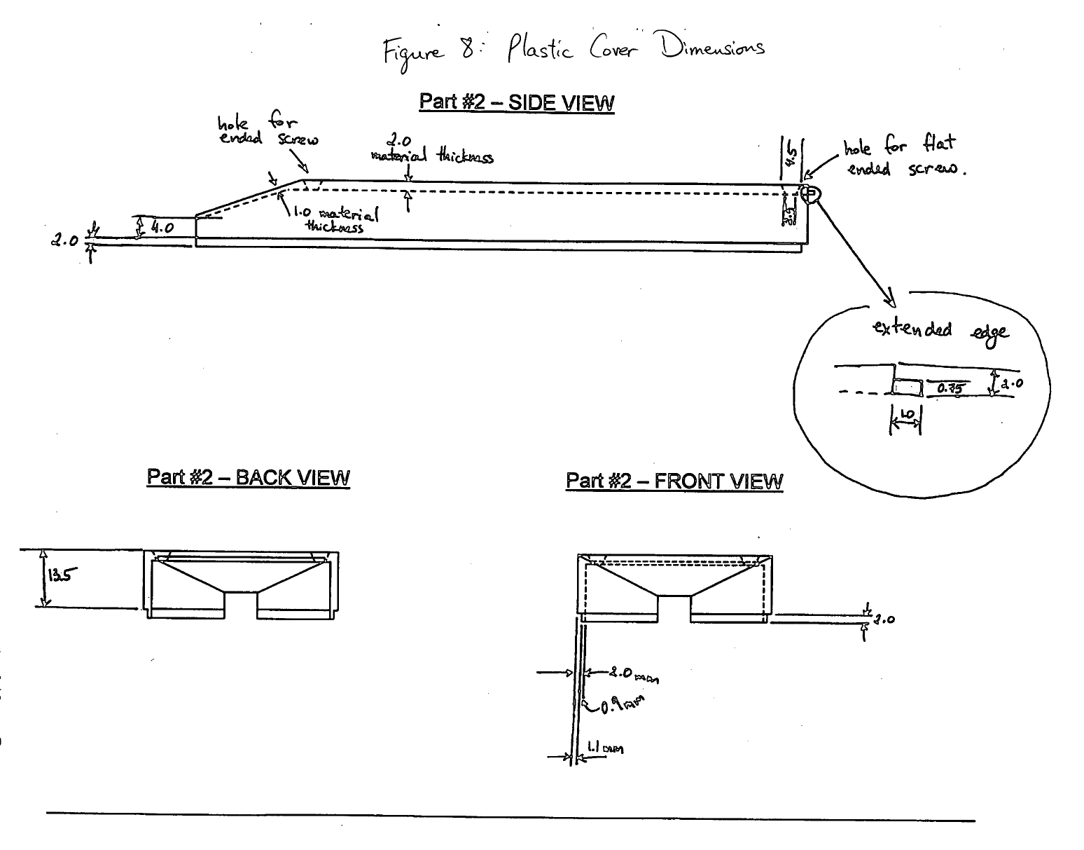

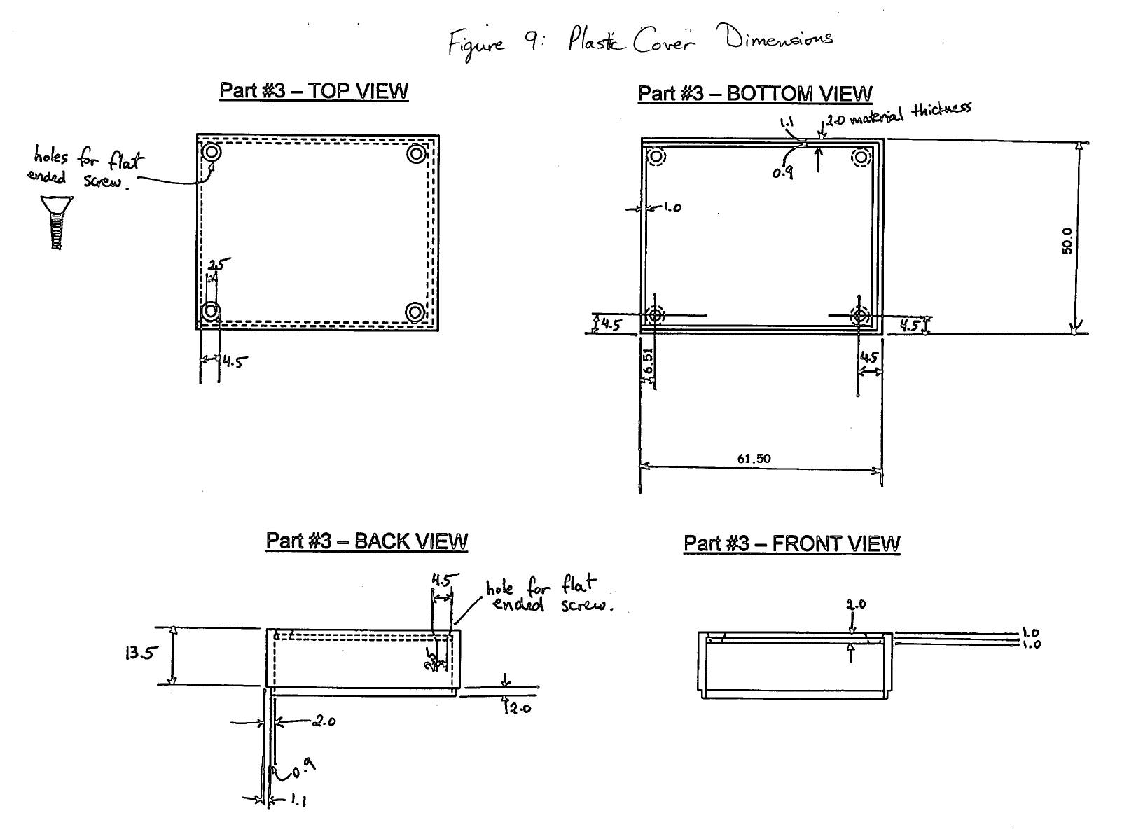

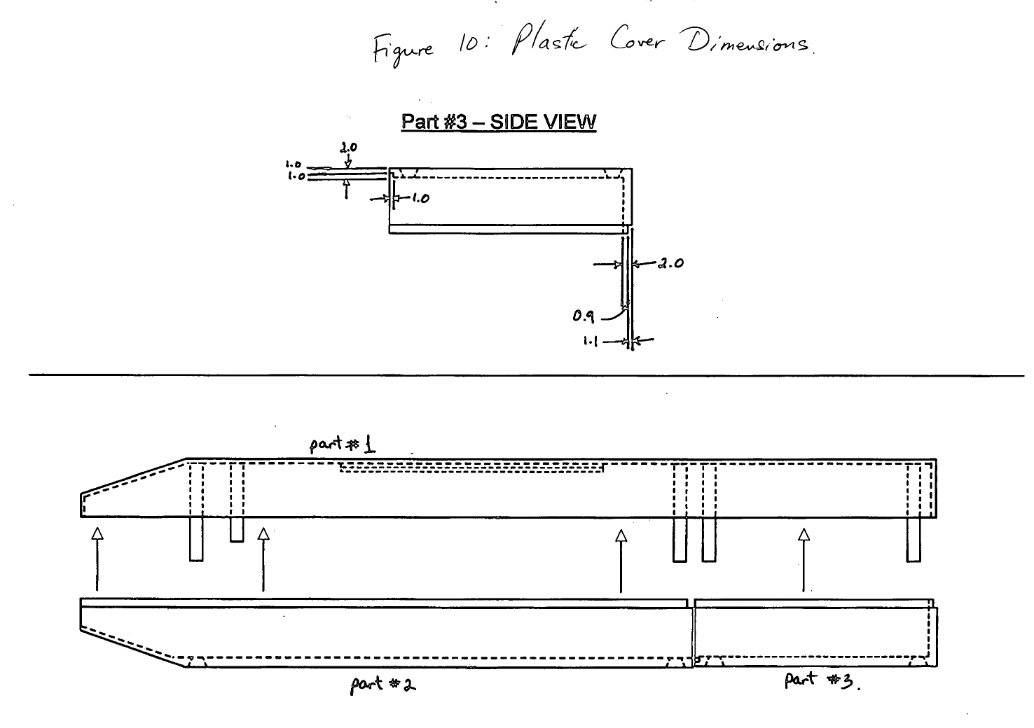

What is claimed is:

1. Device used to measure human skin tone darkness to assist in laser and light based treatments of the skin.
